(19) United States
(12) Patent Application Publication
(10) Pub. No.: US 2015/0297869 A1
(43) Pub. Date: Oct. 22, 2015

(54) SYSTEMS AND METHODS FOR TRANSESOPHAGEAL PROCEDURES USING WIRE GUIDES

(71) Applicant: MICROACCESS INC., Quincy, MA (US)

(72) Inventor: Sameh Mesallum, Boston, MA (US)

(21) Appl. No.: 14/630,261

(22) Filed: Feb. 24, 2015

Related U.S. Application Data
(60) Provisional application No. 61/943,641, filed on Feb. 24, 2014.

(51) Int. Cl. A61M 25/09; A61B 19/00; A61B 17/00

(52) U.S. Cl. CPC A61M 25/09; A61B 17/00234; A61B 19/50; A61M 25/09041; A61M 2205/0272; A61M 2205/52; A61M 2210/105; A61B 2017/00318; A61B 2017/00876; A61B 2017/00243; A61B 2017/00278

(57) ABSTRACT

According to various aspects, systems and methods for delivering a surgical module to a surgical field are provided. According to one embodiment, magnetic fields provided by at least one of the surgical module and a guide wire are manipulated to move the surgical module relative to the guide wire. In some examples, the guide wire is placed specifically to deliver surgical modules having a variety of surgical instruments to a target area with a patient. Once positioned, the surgical modules can execute a variety of surgical procedures.

### RELATED APPLICATIONS

This application claims priority under 35 U.S.C. §119(e) to U.S. Provisional Application Ser. No. 61/943,641 entitled “SYSTEMS AND METHODS FOR TRANSESOPHAGEAL PROCEDURES USING WIRE GUIDES,” filed Feb. 24, 2014, which application is incorporated herein by reference in its entirety.

### BACKGROUND

Providing surgical access to the human heart, the thoracic cavity, the neck structures, the cervical spine, and the dorsal spine has always been difficult and a source of active research. Conventional approaches have sought to leverage advancements in minimally invasive surgery and associated technology to provide a variety of approaches to orthopedic procedures, neurosurgical procedures, and even cardiac procedures. Some conventional approaches have implemented natural pathways to reach surgical targets further minimizing the invasiveness of the intended surgery.

### SUMMARY

It is appreciated that such conventional approaches still fail to provide stable but flexible delivery mechanisms for placing surgical devices within an intended surgical field. Accordingly, provided are systems and methods for delivering surgical instruments to a surgical field following minimally invasive pathways. In one embodiment, a magnetic guide wire is employed, that can be inserted through a natural body orifice to reach a desired surgical location. Surgical modules are deployed using the magnetic guide wire. Interaction between magnetic fields of the guide wire and the surgical modules are configured to drive the surgical modules along the guide wire to position the surgical modules at the surgical field. In some embodiments, the guide wire forms a stable platform from which the surgical modules can operate. In other embodiments, the surgical modules can be guided by the guide wire, and then anchored in position to perform a surgical procedure.

According to some embodiments, provided is a guide wire with fixed or electric coil magnets. Corresponding magnets or electric coils on surgical modules enable the module to traverse the magnetic guide wire by manipulating the polarity and/or intensity of the magnetic fields in the module or in the wire. The interaction between the magnets positioned in the wire and module results in an applied force to move the surgical module in a desired direction (e.g., down the wire, up the wire, or around the wire as needed). According to some embodiments, the surgical modules can include cameras, lights, sensors, and a variety of other surgical devices. For example, the surgical modules can also include scalpels, sensors for sensing and mapping, ablation devices, syringes, probes, suturing devices, lasers, among other options.

According to one aspect, a system for delivering surgical instruments to a surgical field is provided. The system comprises a guide wire, wherein the guide wire is configured to provide magnetic fields along the length of the guide wire; and a surgical module having a channel for movably coupling the surgical module to the guide wire, wherein the surgical module is configured to provide at least one magnetic field, and traverse the guide wire responsive to interactions between the magnetic fields of the guide wire and the at least on magnetic field of the surgical module.

In one embodiment, the system further comprises a control unit configured to manipulate the magnetic fields of at least one of the guide wire and the surgical module. In one embodiment, the control unit is configured to manipulate the magnetic fields to provide movement of the surgical module relative to the guide wire. In one embodiment, the control unit is configured to move the surgical module forward, backward, and rotate the surgical instrument around the guide wire. In one embodiment, the control unit is configured to manipulate the magnetic fields responsive to wireless control signals. In one embodiment, the system further comprises at least one processor operatively connected to a memory, wherein the processor is configured to execute instructions from the memory to position the surgical module at a surgical field within a patient.

In one embodiment, the system further comprises at least one processor operatively connected to a memory, wherein the processor is configured to execute instructions from the memory to perform a surgical procedure according to a predefined program. In one embodiment, the predefined program defines steps executed by the surgical module to perform the surgical procedure. In one embodiment, the predefined program defines steps executed by a plurality of surgical modules to perform the surgical procedure.

According to one aspect, a computer implemented method for delivering surgical instruments to a surgical field is provided. The method comprises inserting a guide wire into a patient; attaching a surgical module to the guide wire, wherein the surgical module includes a channel for movably coupling the surgical module to the guide wire; manipulating magnetic fields produced at least one of the guide wire and the surgical module; and moving the surgical module relative to the guide wire responsive to the act of manipulating the magnetic fields. In one embodiment, the act of inserting the guide wire includes inserting the guide wire into a natural body opening of a patient. In one embodiment, the act of manipulating the magnetic fields is executed by at a control unit configured to manipulate the magnetic fields of at least one of the guide wire and the surgical module. In one embodiment, moving the surgical module relative to the guide wire includes moving the surgical module forward, backwards, and around relative to the guide wire. In one embodiment, the method includes manipulating the magnetic fields responsive to wireless control signals. In one embodiment, the act of moving includes an act of positioning the surgical module at a surgical field within a patient.

In one embodiment, the method further comprises performing, by the surgical module, a surgical procedure. In one embodiment, the surgical procedure is executed according to a predefined program. In one embodiment, the predefined program defines steps executed by at least one surgical module to perform the surgical procedure. In one embodiment, the predefined program defines steps executed by a plurality of surgical modules to perform the surgical procedure.

According to one aspect a non-transitory computer readable medium is provided. The readable medium having stored thereon sequences of instruction for delivering surgical instruments to a surgical field, including instructions that when executed cause at least one processor of a computer system to manipulate magnetic fields produced at at least one of a guide wire and a surgical module; move the surgical module relative to the guide wire responsive to the act of manipulating the magnetic fields; and control, the surgical module, during a surgical procedure.

Still other aspects, embodiments and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Any embodiment disclosed herein may be combined with any other embodiment. References to “an embodiment,” “an example,” “some embodiments,” “some examples,” “an alternate embodiment,” “various embodiments,” “one embodiment,” “at least one embodiment,” “this and other embodiments” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1A illustrates an example guide wire, according to one embodiment;

FIG. 1B illustrates an example guide wire, according to one embodiment;

FIG. 2A illustrates an example guide wire, according to one embodiment;

FIG. 2B illustrates a cross section of an example guide wire, according to one embodiment;

FIG. 2C illustrates an example guide wire, according to one embodiment;

FIG. 3A illustrates an example surgical module according to one embodiment;

FIG. 3B illustrates an example surgical module according to one embodiment;

FIG. 4 illustrates an example surgical module according to one embodiment;

FIG. 5 illustrates an example surgical module including a surgical device according to one embodiment;

FIG. 6 illustrates an example surgical module including surgical components according to one embodiment;

FIG. 7 illustrates an example surgical module including surgical components according to one embodiment;

FIG. 8 illustrates an example surgical module deployed in a patient's GI tract, according to one embodiment;

FIG. 9 illustrates an example surgical module deployed in a patient's thoracic cavity, according to one embodiment;

FIG. 10 illustrates an example surgical module deployed in a patient's blood vessel, according to one embodiment;

FIG. 11 illustrates an example surgical module deployed in a patient's abdomen, according to one embodiment;

FIG. 12 illustrates an example surgical module deployed in a patient's thorax, according to one embodiment;

FIG. 13 illustrates delivery of multiple surgical modules to a target location over multiple guide wires, according to one embodiment;

FIG. 14 illustrates delivery of multiple surgical modules to a target location over a single guide wire, according to one embodiment;

FIG. 15 illustrates delivery of an example surgical module to a patient's vertebral column, according to one embodiment;

FIG. 16 illustrates positioning of an example guide wire and module for a surgical procedure, according to one embodiment;

FIG. 17 illustrates positioning of multiple guide wires and modules for a surgical procedure, according to one embodiment;

FIG. 18 illustrates and example process flow for delivering surgical modules to a target location, according to one embodiment; and

FIG. 19 is a schematic diagram of an exemplary computer system that may be configured to perform processes and functions disclosed herein.

### DETAILED DESCRIPTION

At least some embodiments disclosed herein include apparatus and processes for performing minimally invasive surgery. Further aspects and embodiments include a magnetic guide wire and surgical module that can be used in any surgical procedure. According to some embodiments, a magnetic guide wire provides a platform on which a surgical module can be guided to a surgical field (i.e., a desire site for surgery). The guide wire can include fixed magnets and/or electric coils for generating magnetic fields along the wire. Each surgical module can, likewise, include fixed magnets and/or electrical coils for generating magnetic fields. The respective magnetic fields of the guide wire and the surgical modules are configured to interact to drive the surgical module along the length of the guide wire. According to some embodiments, the surgical module can be driven up and down the length of the guide wire. The ability to traverse the guide wire can allow for fine tune positioning of a surgical module in a surgical field, and in some examples, provide a stable platform from which to perform minimally invasive surgical procedures.

Examples of the methods and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

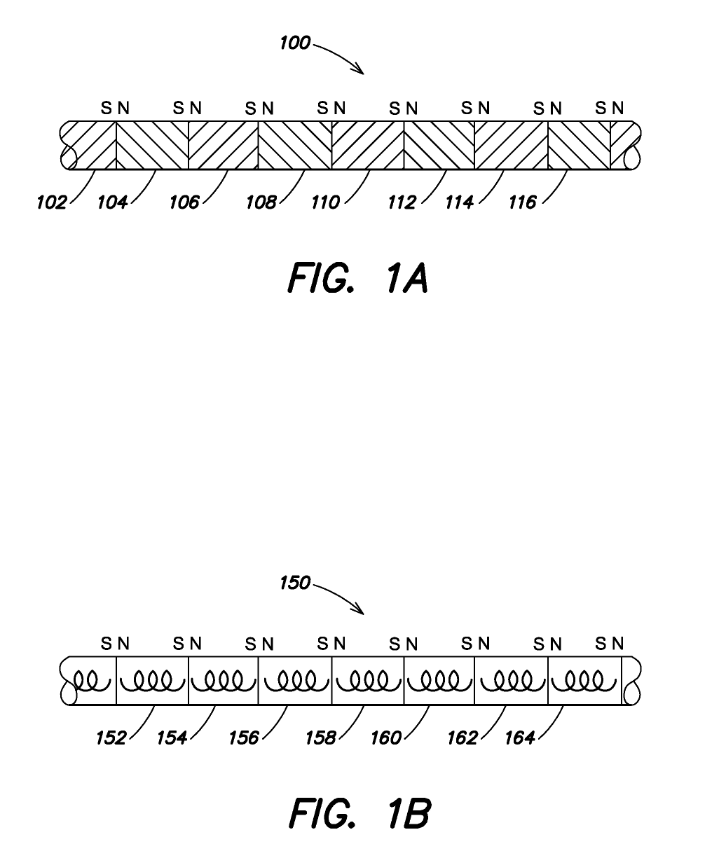

Shown in FIG. 1 is an example embodiment of a guide wire 100. The guide wire 100 includes a plurality of fixed magnets (e.g., 102-116). The fixed magnets can be positioned along the guide wire at regularly spaces intervals. The spacing between the magnets can be configured to accommodate various sizes associated with surgical modules that will traverse the guide wire. According to one embodiment, at each position (e.g., 102-116), one pole of the fixed magnets abuts the opposite pole of the adjacent fixed magnet (e.g., 102 S abuts 104 N). In other embodiments, different spacing of the magnets can be used to provide gaps between the poles of magnets in the guide wire. According to some embodiments, the guide wire is constructed of fixed magnet portions. The fixed magnet portions can be constructed from iron, nickel, cobalt, or alloys capable of ferromagnetism. In other embodiments, the fixed magnet portions can be constructed of materials capable of ferromagnetism. In some embodiments, the fixed magnets are disposed at regular intervals within a mesh structure of a semi-rigid but flexible material that forms the exterior of the guide wire. In further embodiments, the fixed magnets can be integrated into the mesh at regular intervals. In some implementations, magnetic portions can be disposed along the length of the wire and in others the magnetic portions can be disposed perpendicular to the length of the guide wire. Various embodiments incorporate longitudinal and/or perpendicular magnetic portions.

Shown in FIG. 1B is another example embodiment of a guide wire 150. The guide wire 150 includes electromagnetic coils (e.g., 152-164) disposed along the length of the guide wire. Upon application of electricity, the electromagnetic coils 152-164 provide magnetic fields configured to interact with surgical modules traversing the guide wire 150. According to one embodiment, by manipulating the magnetic fields provided by the electromagnetic coils, one or more surgical modules (discussed in greater detail below) can be driven along the length of the guide wire. In some implementations, each of the electromagnetic coils 152-164 can be operated independently by a control unit electrically connected to the coils. In further embodiments, each of the coils shown at 152-164 can include additional coils having different orientations. Selective application of electricity to the additional coils having different orientations allows the control unit and/or an operator to further manipulate movement of, for example, a surgical module along the guide wire. In some implementations, the control unit is configured to vary the polarity and/or intensity of the magnetic field provided by the coils to facilitate fine tune control over the movement of any surgical modules on the guide wire.

In some examples, the surgical modules can also be locked in a fixed position by manipulating the magnetic fields provided by the coils on the guide wire. In further examples, the surgical modules can be driven in any direction along the guide wire, and can also be induced to rotate around the guide wire (e.g., 100 and 150). According to other embodiments, a surgical module that travels the guide wire can be constructed with fixed magnets and/or electromagnetic coils. As discussed in greater detail below, manipulation of the magnetic fields provided by the surgical module (e.g., electromagnetic coils disposed in the module) can, likewise, result in propulsion of the surgical module along the guide wire. Embodiments of the guide wire can include both fixed magnets and electromagnetic coils. FIGS. 1A and 1B illustrate examples of embodiments having fixed magnets or electromagnetic coils. Additional embodiments include both types and different architectures to provide different geometries of magnetic fields along the guide wire.

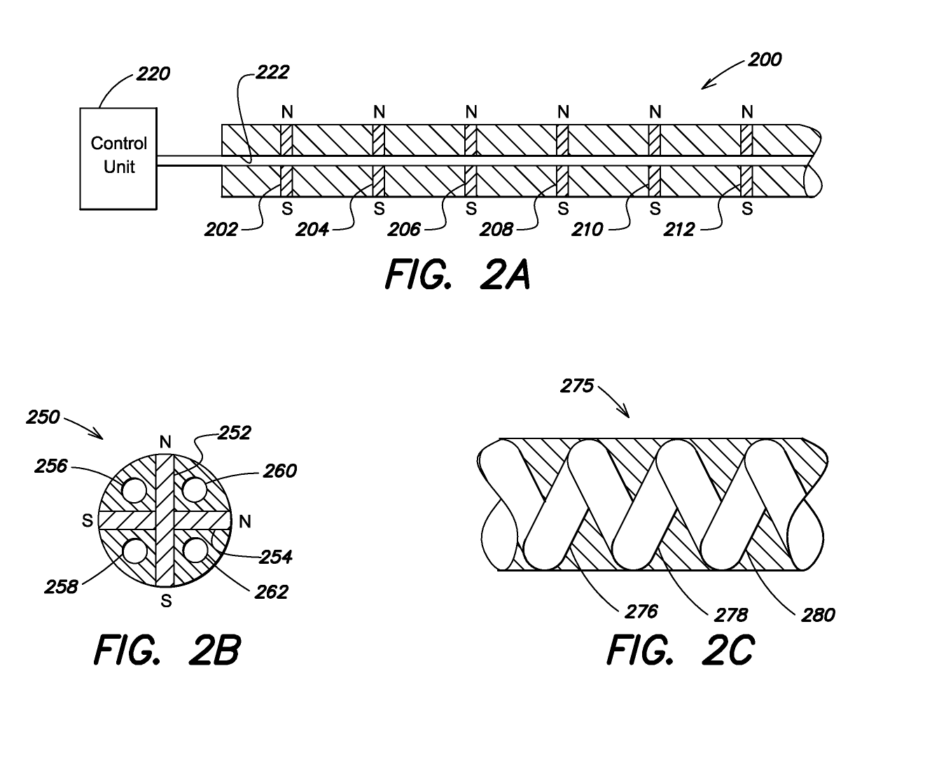

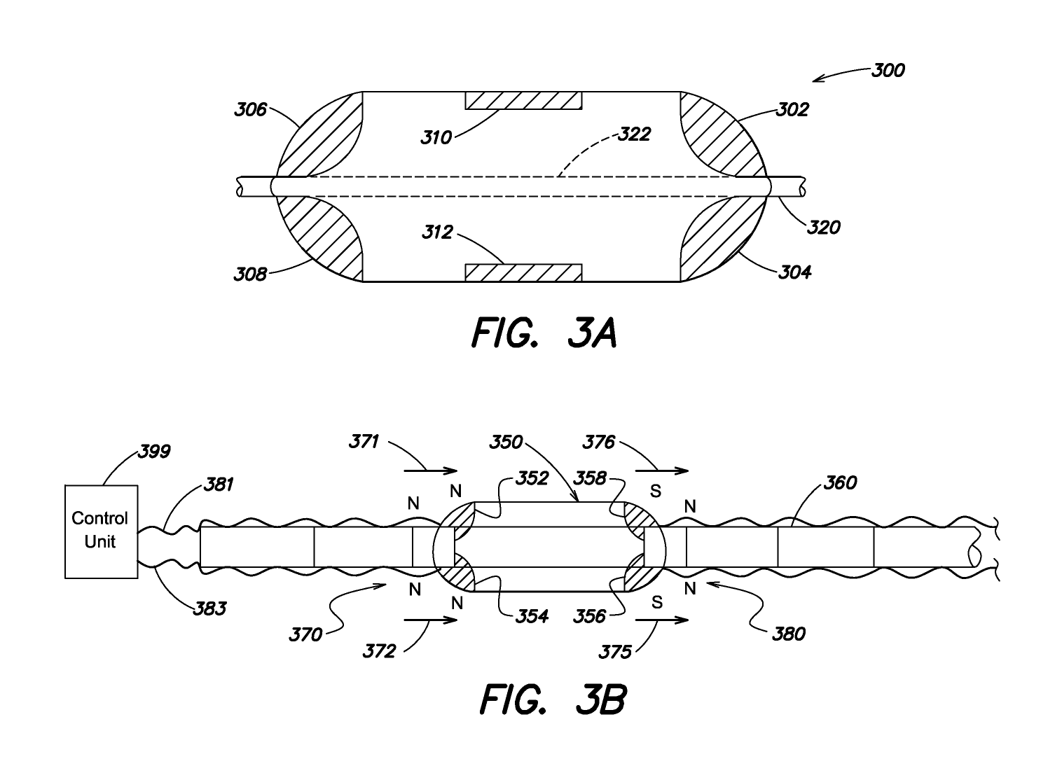

Shown in FIG. 2A is another example embodiment of a guide wire 200. The guide wire 200 includes perpendicular fixed magnets at 202-212. The perpendicular fixed magnets can be disposed at regular intervals along the full length of the guide wire 200. In some implementations, the guide wire 200 can be connected to a cooling unit 220. According to one embodiment, the cooling unit is connected to the guide wire 200 at a cooling lumen 222. The cooling lumen can extend the length of the guide wire. The cooling lumen can be configured to deliver cooling fluid (including e.g., air, liquid, water, liquid nitrogen, or other cooling medium) throughout the guide wire. The cooling unit 220 is configured to maintain the guide wire 200 at a normal body temperature during operation of surgical modules along the guide wire (e.g., 200) and/or during any procedure implemented by use of the guide wire. In some embodiments, the guide wire 200 can include temperature sensors (not shown) configured to provide temperature readings to a control unit and/or the cooling unit 220. In one example, the cooling unit 220 can be configured to maintain normal body temperatures along the guide wire 200 responsive to temperature readings communicated by the temperature sensors. According to some implementations, the cooling unit 220 and the cooling lumen can be implemented in any magnetic guide wire disclosed herein.

Shown in FIG. 2B is another example embodiment of a guide wire 250. FIG. 2B illustrates a cross section view of the guide wire 250. At 252 and 254 two fixed magnets cross the diameter of the guide wire 250, with the north and south poles of 252 and 254 adjacent to each other. In other embodiments, the poles of 252 and 254 can be oriented so the poles alternate around the circumference of the guide wire (e.g., N S N S). The guide wire 250 also includes cooling lumens at 256, 258, 260, and 262 configured to cool the guide wire 250 to normal body temperature. In some examples, the cooling lumens can be connected to a cooling unit at one end of the guide wire (e.g., 250).

FIG. 2C illustrates another example embodiment of a guide wire 275. The guide wire 275 includes fixed magnets along its length (e.g., 276, 278, and 280) that form a spiral array of magnets and respective magnetic fields along the length of the guide wire 275. Interaction between a surgical module and spiral magnets can be manipulated to rotate the surgical module around the guide wire 275 as it moves forward and/or backward along the guide wire.

According to some embodiments, guide wires can include any combination of perpendicular magnets, longitudinal magnets, and spiral magnets. For example, the construction and placement magnets discussed with respect to FIGS. 1A, 1B, 2A, 2B, and 2C can be combined, overlap, and/or be interleaved along the length of embodiments of guide wires. In further embodiments, fixed magnets and electromagnetic coils can be used interchangeably, for example, in the guide wires discussed above. Various implementations of guide wires are configured to provide magnetic fields using any one or more of fixed magnets, perpendicular magnets, spiral magnets, longitudinal magnets and/or electromagnetic coils which interact with magnetic fields present on a surgical module to propel the surgical module along the guide wire.

Shown in FIG. 3 is an example embodiment of a surgical module 300. The surgical module 300 includes a channel 322 extending the length of the module for receiving and traveling along a guide wire 320. The surgical module 300 can include a plurality of magnetic portions. The magnetic fields provided by the magnetic portions interact with respective magnetic fields produced by the guide wire 320. Attraction and/or repulsion between the magnetic fields can be configured to propel the surgical module 300 along the guide wire 320. In some embodiments, the plurality of magnetic portions can be disposed along front or back portion of the module (e.g., 302-304 and 306-308). In some implementations, the magnetic portions 302-308 can be fixed magnets or electromagnetic coils and can include various combinations or fixed magnets with coils.

According to one embodiment, the surgical module 300 can also include additional magnetic portions (e.g., 310 and 312). Positioning of the magnetic portions 310-312 and associated magnetic fields can be configured to control circumferential motion of the surgical module about the guide wire 300. In some examples, control units can be configured to manipulate the polarity of a magnetic field produced by a magnetic portion and/or vary the intensity of the magnetic field produced by a magnetic portion to enable propulsion of the surgical module.

FIG. 3B illustrates manipulation of magnetic fields on at least one of a surgical module 350 and guide wire 360 to propel the module along the guide wire. In some embodiments, a control unit 399 can be configured to provide power to magnetic coils disposed in at least one or both of the surgical module 350 and guide wire 360. For example, the surgical module 350 can include magnetic portions 352-354 and 356-358. The control unit 399 can selectively provide power to the magnetic portions (e.g., 352-358) to produce a desired magnetic field. Interactions between magnetic fields at 370 produce, for example, repulsive forces that propel the surgical module 350 (along lines 371-372). Additionally, the control unit 399 can also power magnetic portions 356-358 through cables 381 and 382 to produce a complementary attractive force at the forward end of the surgical module 350. In some embodiments, the cables 381-382 can follow the exterior of the guide wire or be embedded within the guide wire. In further embodiments, the surgical module 350 can include contacts through which power can be selectively provided to magnetic portions 352-358. In other examples, the surgical module 350 can include an internal power source (e.g., a battery), which can provide power to the magnetic portions.

At 380, respective S and N fields produce an attractive force propelling the surgical module along lines 375-376 to complement the repulsive force from 370. In other embodiments, the control unit 399 can manipulate magnetic fields on either the guide wire or the surgical module through selective power delivery. Additionally, the guide wire and/or surgical module can have multiple electromagnetic coils configured to produce opposite poles of a magnetic field at a given location. By selectively powering the coils, a desired field of a desired polarity can be generated to propel the surgical module (e.g., forward, backward, and around).

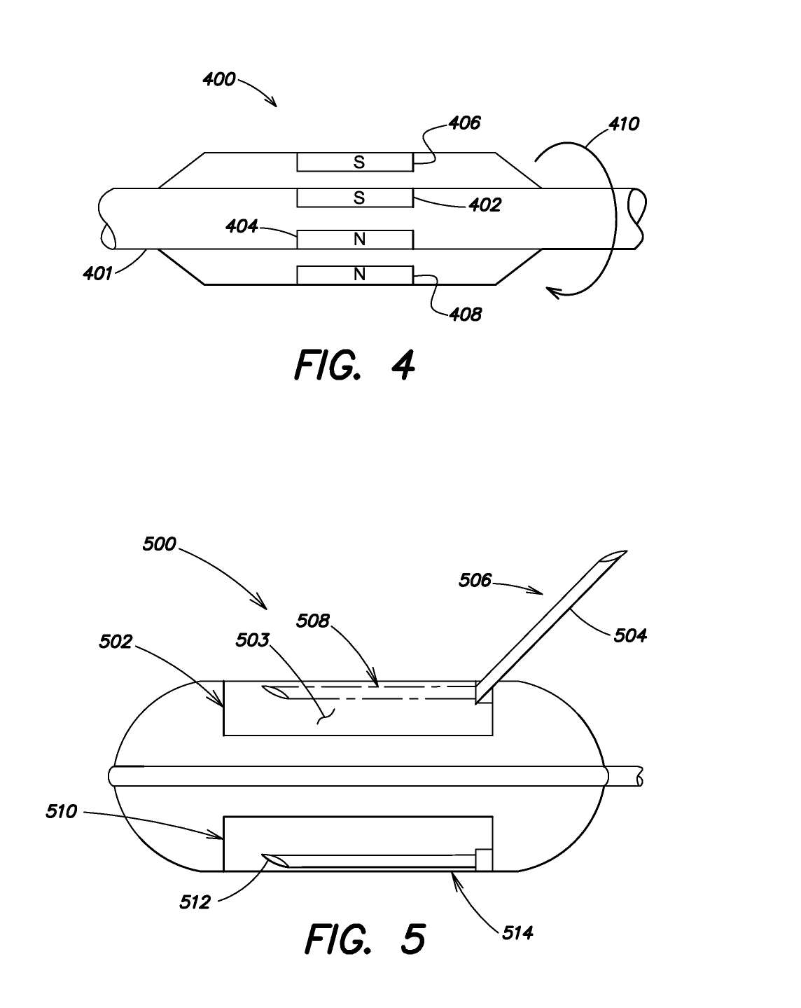

Shown in FIG. 4 is an example of another embodiment of a surgical module 400. As shown, the magnetic fields provided by the surgical module 400 and the guide wire 401 result in a rotational force on the surgical module 400 in the direction of arrow 410. In some embodiments, the magnetic fields provided by magnetic portions 402-408 can be manipulated to rotate the surgical module 400 around the guide wire 401 in either direction.

According to one aspect, the ability to provide fine tune control over motion in any direction (e.g., forward, backward, and around) enables delivery of surgical modules having a variety of surgical instruments through minimally invasive pathways. Fine tune control over the motion of the surgical module enables execution of complex surgical procedures. In some settings, multiple surgical modules can be used in concert and/or execute a common or collective program to perform complex surgical procedures in locations throughout the human body. Shown in FIG. 5, is an example embodiment of a surgical module 500. For the purposes of clarity some of the components (e.g., magnetic portions) are not shown. The surgical module 500 includes an injection component 502. The injection component 502 can include a reservoir 503 for holding a therapeutic material (e.g., drug, dye, antibiotics, chemotherapy agent, etc.). The injection component 502 includes a delivery device (e.g., an injection needle 504). In some embodiments, the injection component 502 can transition the delivery device between a deployed state 506 and an un-deployed state 508. In some examples, the injection component 502 can be responsive to control signals provide by or to the surgical module to transition between deployed and un-deployed states. In one example, a control unit can provide command signals to deploy the delivery device. The injection component 502 can also include mechanical plungers or other devices to force the therapeutic material from the reservoir and into a target site or organ. Multiple injection components can be included in the surgical module (e.g., at 510 is a second injection component). At 510 shown is a second injection component with a respective delivery device 512 in an un-deployed state 514.

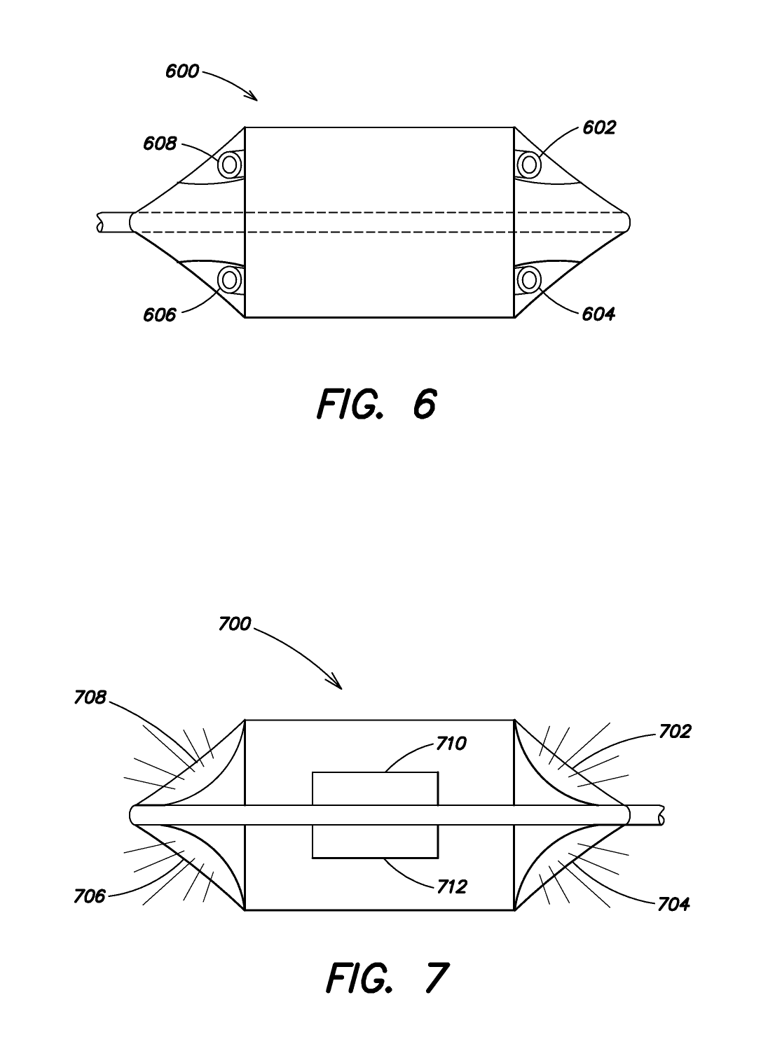

FIG. 6 illustrates a surgical module 600 having a plurality of camera components at 602, 604, 606, and 608. The surgical module can include one or more internal batteries (e.g., 610). The batteries can be used to power the camera components and any lights (e.g., LED light sources) included in the camera components. The camera components shown in module 600 can be included in other surgical modules (e.g., 400 and 500), and other components illustrated in other surgical modules can likewise be included in 600 (e.g., injection component 502 of FIG. 5). FIG. 7 illustrates lights components 702-708 on a surgical module 700. The light components can be constructed of LED light assemblies. In some embodiments, the surgical module 700 can include internal batteries 710 and 712 to power various surgical components (e.g., 702-708) within the surgical module. In other embodiments, power can be supplied through cables running along or through the guide wire. In yet other embodiments, both battery and cabling can supply power to the surgical module and/or surgical components.

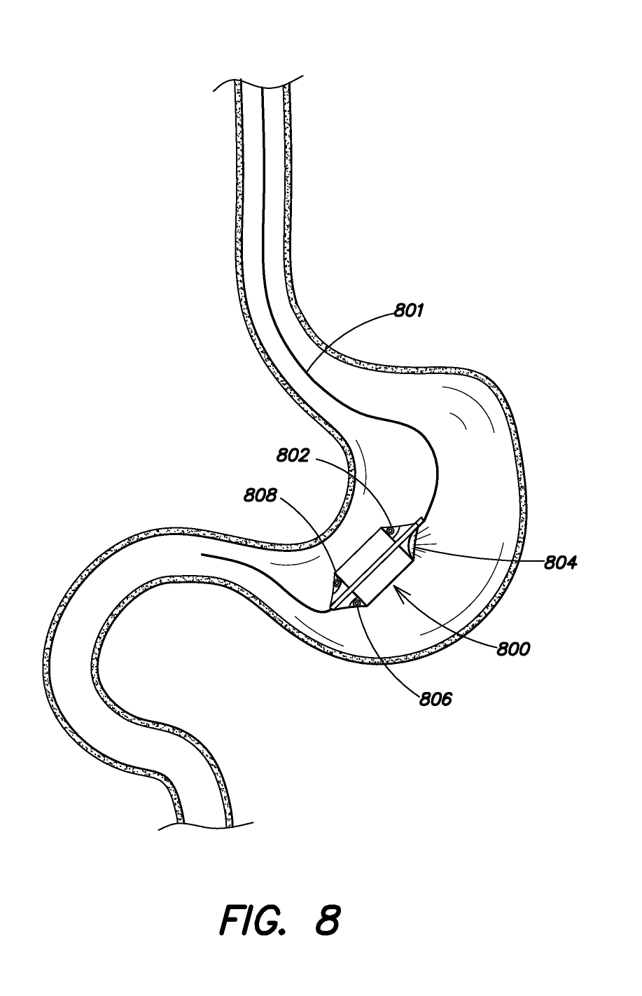

FIG. 8 shows a surgical module 800 deployed in a patient's GI track. The surgical module has traversed the guide wire 801 to arrive at a field of interest. In this example, the surgical module 800 includes front facing camera 802 and light 804 components and backward facing camera 806 and light 808 components. According to some embodiments, the guide wire (e.g., 801) can be deployed within a patient through a natural body opening. In one example, the patient can swallow a capsule containing the guide wire, which deploys as the capsule travels along the patient's digestive tract. Once the guide wire is deployed or at least partially extended, a surgical module can be coupled to the guide wire. As discussed above, magnetic field interactions between the surgical module and the guide wire enable movement of the module 800 to any desired position along the guide wire 801. The pairs of cameras and lights (e.g., 802-804 and 806-808) can be used, for example, to image the patient's GI track. In some embodiments, once a surgical process is complete the surgical module and/or guide wire can be allowed to pass through the patient's digestive tract exiting the patient's body through normal bodily functions.

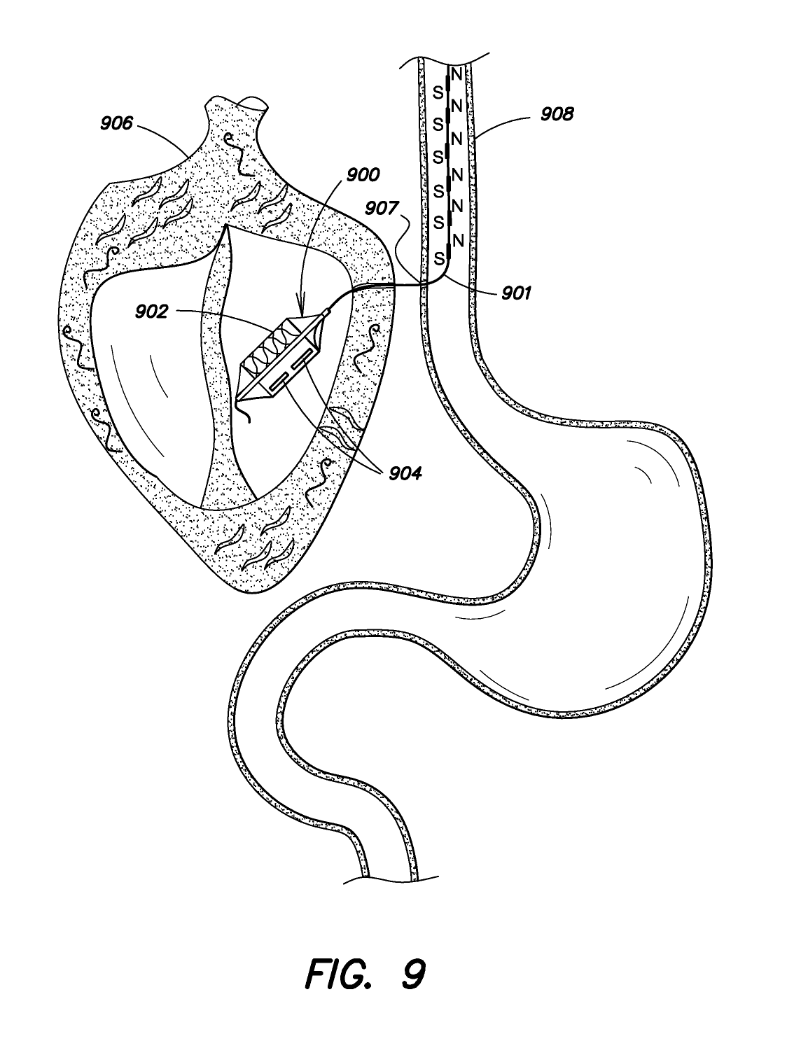

FIG. 9 shows another embodiment of a surgical module 900 and another approach for accessing an area of a patient's anatomy. The surgical module 900 can be deployed within the patient's esophagus 908. The guide wire is extended through the esophagus at 907 into the patient's thoracic cavity. The surgical module 900 can be configured to perform a number of operations within the thoracic cavity. In one example, the surgical module 900 includes an ablation array at 902. The ablation array 902 is configured to ablate tissue in response to power/control signals delivered to the ablation array 902. In some embodiments, the power/control signal can include RF signals that cause the ablation array 902 to ablate proximate tissue.

In further examples, the surgical module 900 can include sensors at 904. The sensors can be configured to map the thoracic cavity, the heart, specific cardiac structure, and/or other regions within the thoracic cavity. In some embodiments, mapping and ablations can be done together by the surgical module 900. In other embodiments, multiple surgical modules can be used to provide individualized functions.

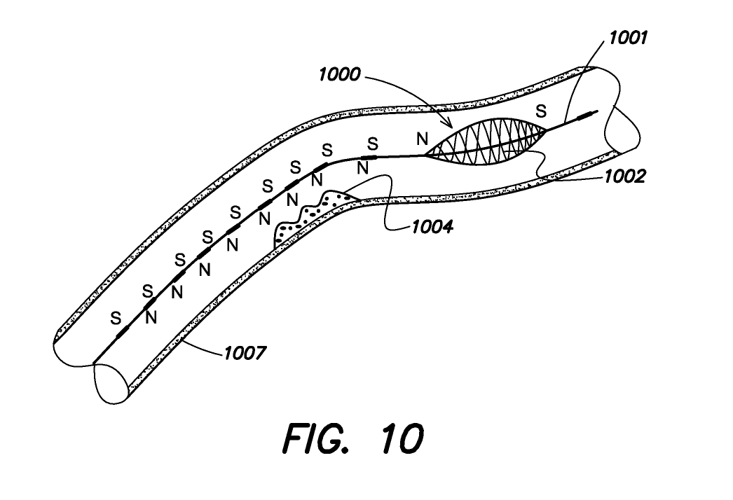

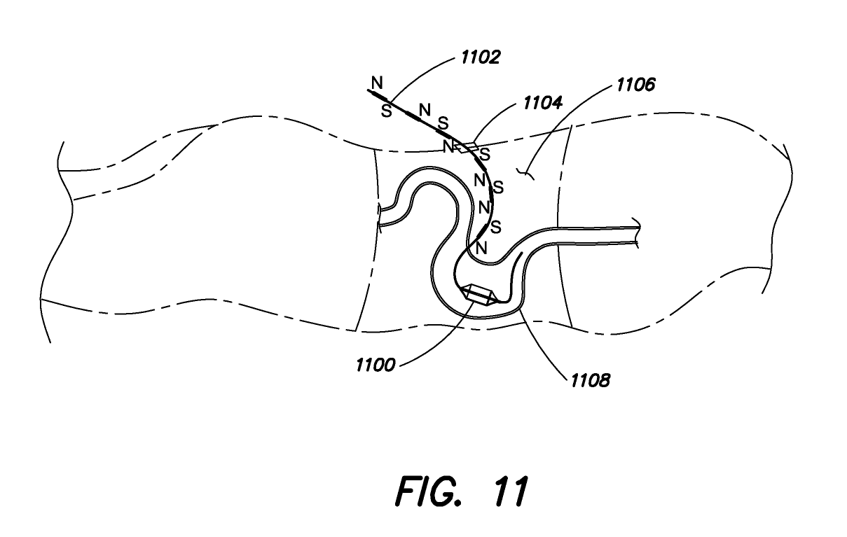

FIG. 10 shows another embodiment of a surgical module 1000 and a surgical function performed. According to one embodiment, the surgical module 1000 can be configured to carry and place a stent 1002 within at target area of a patient's anatomy. For example, the surgical module 1000 can be directed to an obstruction in a blood vessel via a guide wire 1001. The guide wire can be positioned in advance of deploying the surgical module 1000, or in some examples, the guide wire 1001 and module 1000 can be introduced together. Shown at 1004 is an obstruction. The surgical module 1000 is deployed into position proximate to the obstruction and the stent 1002 is placed within the patient to resolve and/or ameliorate the obstruction. In some embodiments, the surgical module 1000 and/or stent 1002 are specially configured to resolve intra-coronary obstructions (e.g., atheroma) in, for example, a coronary blood vessel 1007. In other embodiments, various guide wire and surgical module combinations can be employed to deliver stents to other locations within a patient. For example, the surgical modules can accommodate a variety of stents for a variety of locations in the body.

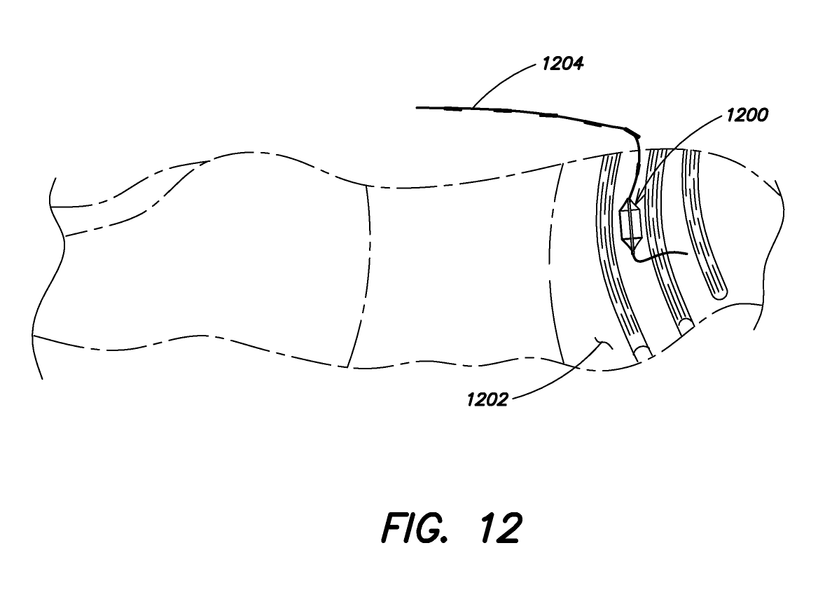

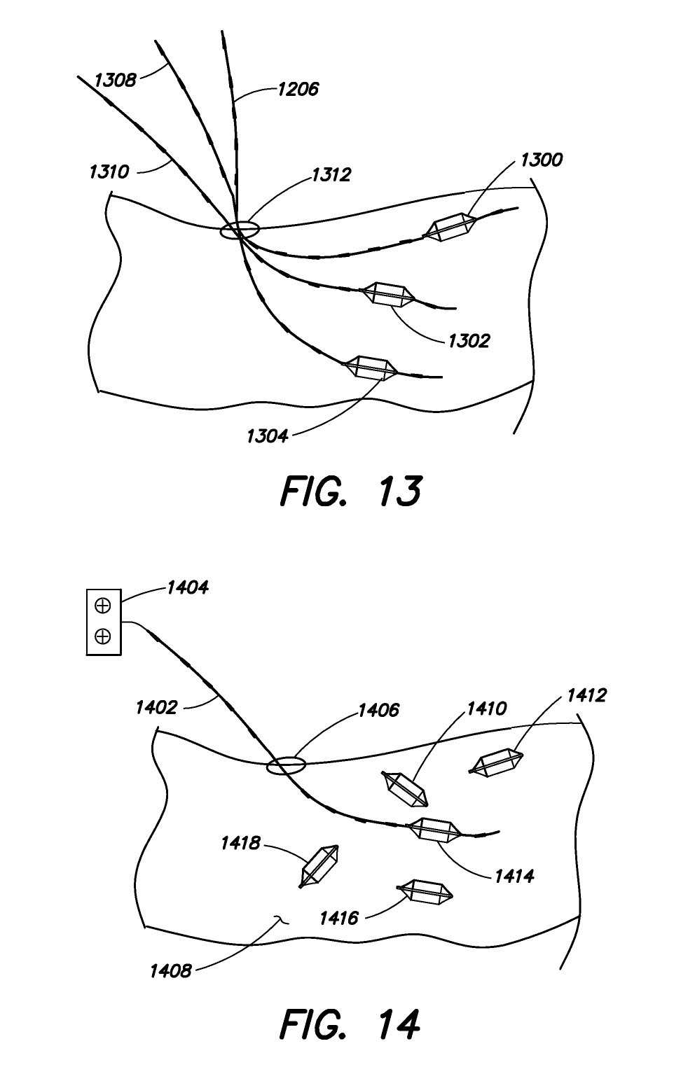

FIG. 11 shows an embodiment of a surgical module 1100 being introduced to a target area of a patient's anatomy by a trans-cutaneous abdominal wall approach (e.g., laparotomy). The surgical module 1100 is configured to follow a guide wire 1102 through an abdominal wall incision 1104 into the patient's intraparietal cavity 1106 of the patient's abdomen 1108. FIG. 12 illustrates delivery of a surgical module 1200 to a patient's thoracic cavity 1202 by a thoracostomy approach. A guide wire 1204 is introduced into the thoracic cavity 1202 via an incision in the thoracic wall. The surgical module 1200 can be driven along the guide wire 1204 to a target area within the thoracic cavity 1202 where a variety of surgical procedures can be performed (e.g., ablation, mapping, therapeutic delivery, etc.). FIG. 13 illustrates introduction of multiple surgical modules 1300, 1302, and 1304 over a plurality of guide wires 1306, 1308, and 1310. The guide wires 1306-1310 can be directed into any body cavity of the patient through an incision (e.g., 1312) in the body cavity wall or lining. Once the modules have been deployed, the modules can be operated individually and/or in concert to perform various surgical procedures. In some embodiments, the surgical modules can operate together to perform a complex surgical procedure. In further embodiments, the modules 1300-1306 can be pre-programmed to execute a surgical procedure including a number of operative steps and/or using a number of different surgical devices (e.g., scalpel, laser, suture, ablation, and/or drug delivery devices, among other options).

FIG. 14 illustrates delivery of multiple surgical modules over a single guide wire 1402. Positioning of the single guide wire 1402 can be controlled via a motion control unit 1404 configured to manipulate the movement and direction of the guide wire 1402. The guide wire 1402 can be introduced through an opening 1406 made by way of incision into a body cavity 1408. Once a surgical module (e.g., 1410-1418) has obtained a desired position and/or been directed to a desired position, the module can be configured to anchor itself in adjacent tissue. In some embodiments, the surgical modules (e.g., 1410-1418) can be configured to clamp to adjacent tissue, can be adhered to a specific location, suture itself to adjacent tissue, etc. In some examples, the guide wire 1402 can then be withdrawn to leave the surgical module in position. In other embodiments, the surgical module can be propelled off of the guide wire. The guide wire 1402 can then be transitioned to a new location for positioning the next surgical module (e.g., 1410-1418).

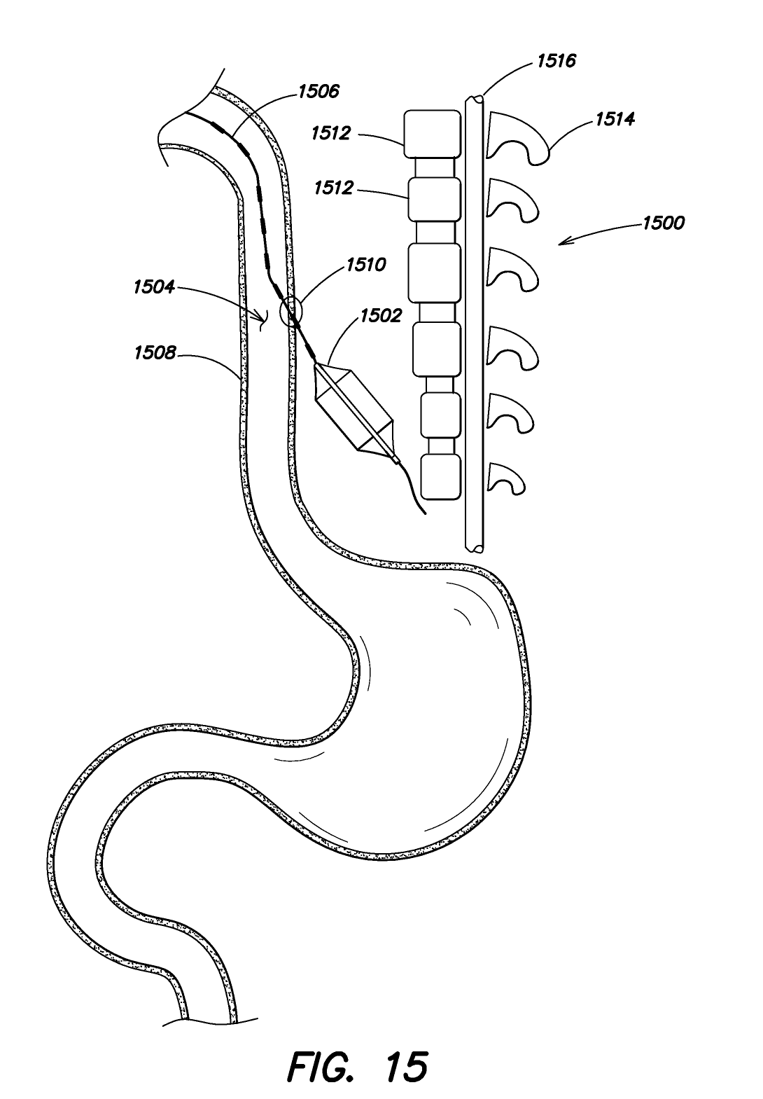

FIG. 15 illustrates a transesophageal surgical approach to a patient's vertebral column 1500. Various embodiments of surgical modules (e.g., 1502) can be configured to perform surgical operations on the patient's vertebral column 1500, including, for example, disk repair, fusion, among other procedures. As shown, the surgical module 1502 can be introduced to the surgical field through a patient's esophagus 1504. In one example, a guide wire 1506 is extended along the esophagus and through the esophageal wall 1508 at an incision 1510. In some implementations, the incisions can be covered by a liner to facilitate providing a sterile field during execution of a surgical procedure via surgical module 1502. Various liner implementations are discussed with respect to application Ser. No. 12/462,268, entitled “Methods and apparatus for transesophageal microaccess surgery,” published as US2010-0036197, which application is incorporated herein by reference in its entirety. Use of liners within the esophagus can provide a sterile operating field. Guide wires and/or surgical modules can be directed through such liners to insure sterility. Maintaining sterility of the operating environment can be especially important when performing surgery on the vertebral column 1500, vertebrae 1512, lamina 1514, and/or the spinal cord 1516.

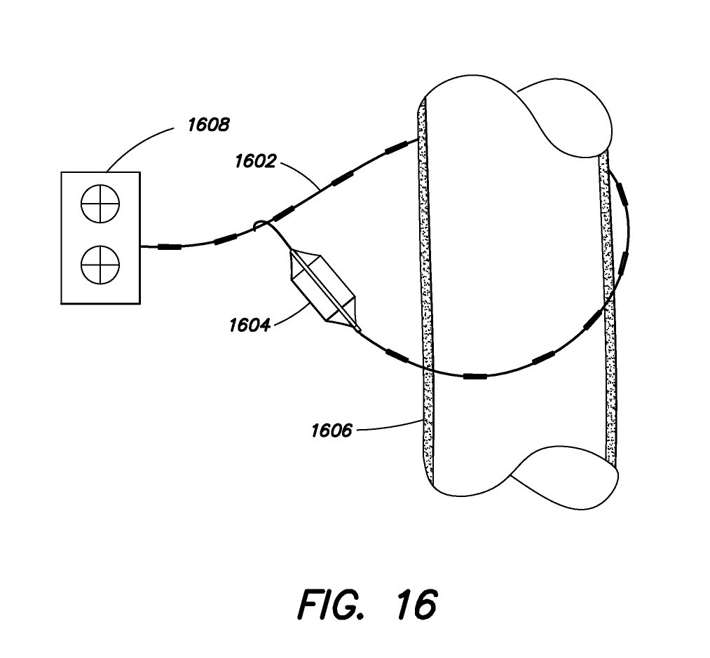

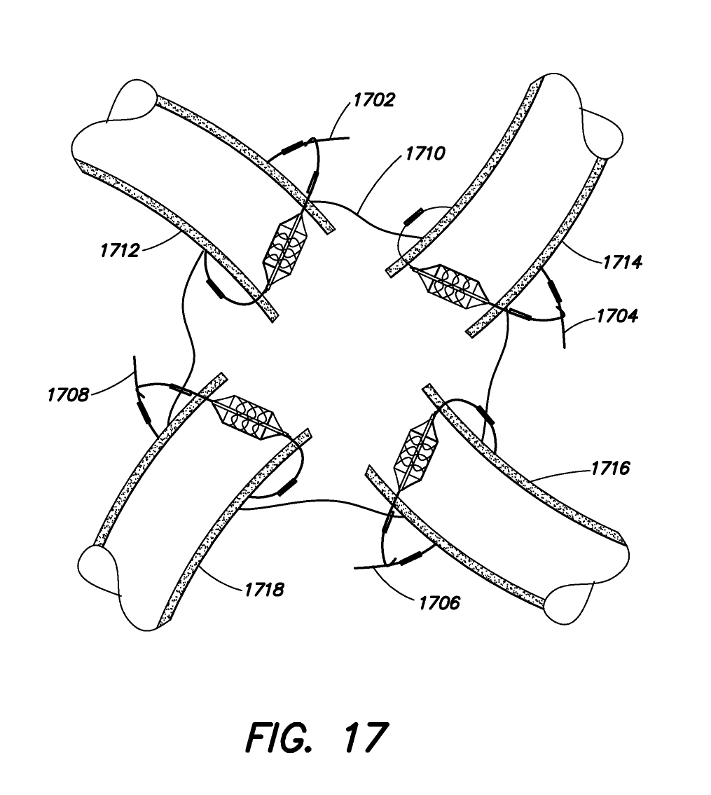

FIG. 16 illustrates another procedure that can be executed with a guide wire 1602 and one or more surgical modules (e.g., 1604). In FIG. 16, a patient's blood vessel is surrounded by a guide wire 1602. In one embodiment, the guide wire 1602 is attached and/or deployed using a control unit 1608 configured to control movement of the guide wire during installation. In some implementations, the control unit 1608 can be programmed to surround, for example, the blood vessel 1606. Alternatively the guide wire can be positioned manually (e.g., by a physician). Once the guide wire is in place, one or more surgical modules (e.g., 1604) can be deployed to execute one or more surgical procedures on the blood vessel 1606. Similarly, the control unit 1608 or physician can deploy the guide wire 1602 around a target organ, positioning one or more surgical modules around the target organ for executing a surgical procedure.

FIG. 17 illustrates another procedure that can be executed according to various embodiments. A plurality of guide wires (e.g., 1702, 1704, 1706, and 1708) are deployed around respective pulmonary veins (e.g., 1712, 1714, 1716, and 1718) of the patient's heart, and in particular, the patient's left atrium 1710. According to one embodiment, the surgical modules 1712-1718 can be configured with ablation components (e.g., RF ablation and/or cryo-based ablation). Each module can be programmed to ablate the pulmonary tissue independently, consecutively, and/or in conjunction, depending on a specified program or control signal.

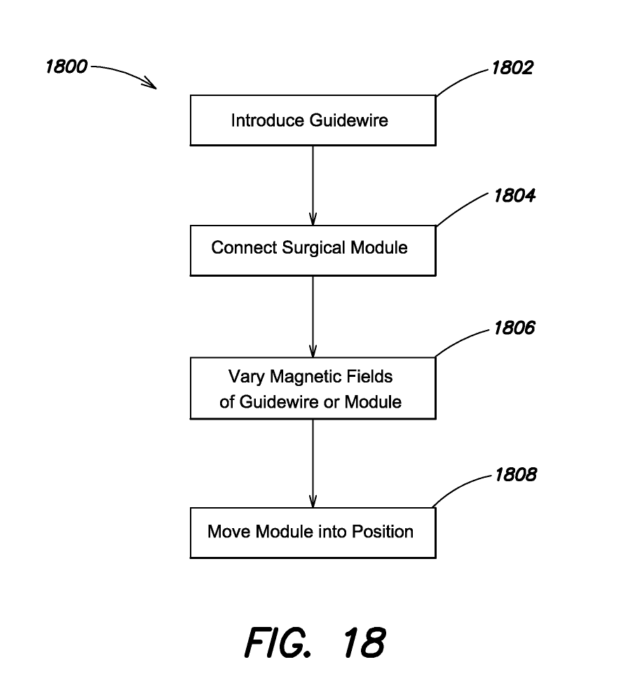

Shown in FIG. 18 is an example process 1800 for positioning a surgical module at a target area within a patient's anatomy. The process 1800 begins at 1802 with introduction of a guide wire into the patient. The guide wire can be introduced through natural body openings reducing the impact of any surgical procedure. For example, the guide wire can be introduced through the patient's mouth. In some examples, the patient may be asked to swallow the guide wire. In other examples, the guide wire can be introduced through the patient's mount, nose, anus, vagina, and/or ear to position the guide wire at a surgical field while providing minimal invasiveness. In other implementations, the guide wire can be introduced at 1802 through surgical openings, as well as natural body openings. The process 1800 continues at 1804 with connecting a surgical module to the guide wire. Although in some examples, the module can already be connected to the guide wire at or before 1802. As discussed above, the guide wire and surgical modules are each configured to provide magnetic fields that interact. At 1806, the magnetic fields of one or both of the guide wire and surgical module are varied. Variation in the intensity and/or polarity of the magnetic fields results in motion of the surgical module. By selectively varying the magnetic fields, attraction and/or repulsion forces can be utilized to move the surgical module into position at a surgical field (e.g., at 1808).

In some implementations, the guide wire can provide a stable platform for the surgical module to executed surgical procedures. For example, the surgical module can include a scalpel for excising lesions, a biopsy needle for capturing tissue samples, among other options. The surgical modules can operate together to perform complex surgeries. In some embodiments, the surgical modules can execute pre-programmed procedures, as well as dynamic instructions provided by an operator. In further embodiments, pre-programmed routines can be accompanied and/or augmented by dynamic operator instructions.

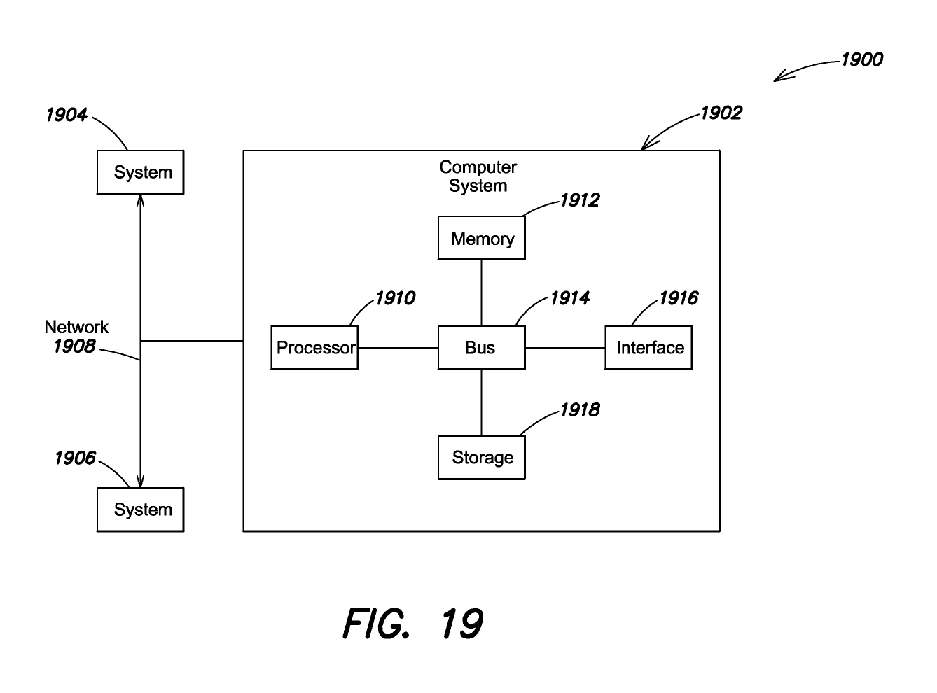

Shown in FIG. 19 is an example special purpose computer system on which various surgical procedures can be programmed and/or executed. In some embodiments, a control unit can include a computer system (e.g., 1900 and/or 1902) which is specially configured to operate surgical modules, determine surgical module positions, capture and render internal anatomy images, manipulate magnetic fields, execute surgical programs, etc. In further embodiments, surgical modules can also include a special purpose computer system (e.g., 1900 and/or 1902). The surgical modules can move themselves into a surgical field within a patient based on programming residing on the surgical module, including functions to manipulate magnetic field to induce movement, detect current position, and induce further movement until a desired location is reached. In some implementations, the programming associated with a plurality of surgical modules can be synchronized and/or include dependent execution of a surgical procedure across the plurality of surgical modules.

Various aspects and functions described herein may be implemented as specialized hardware or software components executing in one or more special purpose computer systems. There are many examples of computer systems that are currently in use. These examples include, among others, network appliances, personal computers, workstations, mainframes, networked clients, servers, media servers, application servers, database servers and web servers. Other examples of computer systems may include mobile computing devices, such as cellular phones and personal digital assistants, and network equipment, such as load balancers, routers and switches. Further, aspects may be located on a single computer system or may be distributed among a plurality of computer systems connected to one or more communications networks.

For example, various aspects and functions may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Consequently, examples are not limited to executing on any particular system or group of systems. Further, aspects and functions may be implemented in software, hardware or firmware, or any combination thereof. Thus, aspects and functions may be implemented within methods, acts, systems, system elements and components using a variety of hardware and software configurations, and examples are not limited to any particular distributed architecture, network, or communication protocol.

Referring to FIG. 19, there is illustrated a block diagram of a distributed special purpose computer system 1900, in which various aspects and functions are practiced. As shown, the distributed computer system 1900 includes one more special purpose computer systems that exchange information. More specifically, the distributed computer system 1900 includes computer systems 1902, 1904 and 1906. As shown, the computer systems 1902, 1904 and 1906 are interconnected by, and may exchange data through, a communication network 1908.

In some embodiments, the network 1908 may include any communication network through which computer systems may exchange data. To exchange data using the network 1908, the computer systems 1902, 1904 and 1906 and the network 1908 may use various methods, protocols and standards, including, among others, Fibre Channel, Token Ring, Ethernet, Wireless Ethernet, Bluetooth, IP, IPV6, TCP/IP, UDP, DTN, HTTP, FTP, SNMP, SMS, MMS, SS7, JSON, SOAP, CORBA, REST and Web Services. To ensure data transfer is secure, the computer systems 1902, 1904 and 1906 may transmit data via the network 1908 using a variety of security measures including, for example, TLS, SSL or VPN. While the distributed computer system 1900 illustrates three networked computer systems, the distributed computer system 1900 is not so limited and may include any number of computer systems and computing devices, networked using any medium and communication protocol.

As illustrated in FIG. 19, the computer system 1902 includes a processor 1910, a memory 1912, a bus 1914, an interface 1916 and data storage 1918. To implement at least some of the aspects, functions and processes disclosed herein, the processor 1910 performs a series of instructions that result in manipulated data. The processor 1910 may be any type of processor, multiprocessor or controller. Some exemplary processors include commercially available processors such as an Intel Xeon, Itanium, Core, Celeron, or Pentium processor, an AMD Opteron processor, a Sun UltraSPARC or IBM Power5+ processor and an IBM mainframe chip. The processor 1910 is connected to other system components, including one or more memory devices 1912, by the bus 1914.

The memory 1912 stores programs and data during operation of the computer system 1902. Thus, the memory 1912 may be a relatively high performance, volatile, random access memory such as a dynamic random access memory (DRAM) or static memory (SRAM). However, the memory 1912 may include any device for storing data, such as a disk drive or other non-volatile storage device. Various examples may organize the memory 1912 into particularized and, in some cases, unique structures to perform the functions disclosed herein. These data structures may be sized and organized to store values for particular data and types of data.

Components of the computer system 1902 are coupled by an interconnection element such as the bus 1914. The bus 1914 may include one or more physical busses, for example, busses between components that are integrated within a same machine, but may include any communication coupling between system elements including specialized or standard computing bus technologies such as IDE, SCSI, PCI and InfiniBand. The bus 1914 enables communications, such as data and instructions, to be exchanged between system components of the computer system 1902.

The computer system 1902 also includes one or more interface devices 1916 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 1902 to exchange information and to communicate with external entities, such as users and other systems.

The data storage 1918 includes a computer readable and writeable nonvolatile, or non-transitory, data storage medium in which instructions are stored that define a program or other object that is executed by the processor 1910. The data storage 1918 also may include information that is recorded, on or in, the medium, and that is processed by the processor 1910 during execution of the program. More specifically, the information may be stored in one or more data structures specifically configured to conserve storage space or increase data exchange performance.

The instructions stored in the data storage may be persistently stored as encoded signals, and the instructions may cause the processor 1910 to perform any of the functions described herein. The medium may be, for example, optical disk, magnetic disk or flash memory, among other options. In operation, the processor 1910 or some other controller causes data to be read from the nonvolatile recording medium into another memory, such as the memory 1912, that allows for faster access to the information by the processor 1910 than does the storage medium included in the data storage 1918. The memory may be located in the data storage 1918 or in the memory 1912, however, the processor 1910 manipulates the data within the memory, and then copies the data to the storage medium associated with the data storage 1918 after processing is completed. A variety of components may manage data movement between the storage medium and other memory elements and examples are not limited to particular data management components. Further, examples are not limited to a particular memory system or data storage system.

Although the computer system 1902 is shown by way of example as one type of computer system upon which various aspects and functions may be practiced, aspects and functions are not limited to being implemented on the computer system 1902 as shown in FIG. 19. Various aspects and functions may be practiced on one or more computers having a different architectures or components than that shown in FIG. 19. For instance, the special purpose computer system 1902 may include specially programmed, special-purpose hardware, such as an application-specific integrated circuit (ASIC) tailored to perform a particular operation disclosed herein. While another example may perform the same function using a grid of several special purpose computing devices running MAC OS System X with Motorola PowerPC processors and several specialized computing devices running proprietary hardware and operating systems.

The computer system 1902 may be a computer system including an operating system that manages at least a portion of the hardware elements included in the computer system 1902. In some examples, a processor or controller, such as the processor 1910, executes an operating system. Examples of a particular operating system that may be executed include a Windows-based operating system, such as, Windows NT, Windows 2000 (Windows ME), Windows XP, Windows Vista or Windows 7 or 8 operating systems, available from the Microsoft Corporation, a MAC OS System X operating system available from Apple Computer, one of many Linux-based operating system distributions, for example, the Enterprise Linux operating system available from Red Hat Inc., a Solaris operating system available from Sun Microsystems, or a UNIX operating systems available from various sources. Many other operating systems may be used, and examples are not limited to any particular operating system.

The processor 1910 and operating system together define a computer platform for which application programs in high-level programming languages are written. These component applications may be executable, intermediate, bytecode or interpreted code which communicates over a communication network, for example, the Internet, using a communication protocol, for example, TCP/IP. Similarly, aspects may be implemented using an object-oriented programming language, such as .Net, SmallTalk, Java, C++, Ada, C# (C-Sharp), Objective C, or Javascript. Other object-oriented programming languages may also be used. Alternatively, functional, scripting, or logical programming languages may be used.

Additionally, various aspects and functions may be implemented in a non-programmed environment, for example, documents created in HTML, XML or other format that, when viewed in a window of a browser program, can render aspects of a graphical-user interface or perform other functions.

Further, various examples may be implemented as programmed or non-programmed elements, or any combination thereof. For example, a web page may be implemented using HTML while a data object called from within the web page may be written in C++. Thus, the examples are not limited to a specific programming language and any suitable programming language could be used. Accordingly, the functional components disclosed herein may include a wide variety of elements, e.g. specialized hardware, executable code, data structures or objects, that are configured to perform the functions described herein.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user mode application) or in a commonly shared data structure (such as an application registry that is defined by an operating system). In addition, some examples provide for both system and user interfaces that allow external entities to modify the parameters and thereby configure the behavior of the components.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

What is claimed is:

1. A system for delivering surgical instruments to a surgical field, the system comprising:
a guide wire, wherein the guide wire is configured to provide magnetic fields along the length of the guide wire; and
a surgical module having a channel for movably coupling the surgical module to the guide wire, wherein the surgical module is configured to:
provide at least one magnetic field, and
traverse the guide wire responsive to interactions between the magnetic fields of the guide wire and the at least on magnetic field of the surgical module.

2. The system according to claim 1, further comprising a control unit configured to manipulate the magnetic fields of at least one of the guide wire and the surgical module.

3. The system according to claim 2, wherein the control unit is configured to manipulate the magnetic fields to provide movement of the surgical module relative to the guide wire.

4. The system according to claim 3, wherein the control unit is configured to move the surgical module forward, backward, and rotate the surgical instrument around the guide wire.

5. The system according to claim 2, wherein the control unit is configured to manipulate the magnetic fields responsive to wireless control signals.

6. The system according to claim 1, wherein the system further comprises at least one processor operatively connected to a memory, wherein the processor is configured to execute instructions from the memory to position the surgical module at a surgical field within a patient.

7. The system according to claim 1, wherein the system further comprises at least one processor operatively connected to a memory, wherein the processor is configured to execute instructions from the memory to perform a surgical procedure according to a predefined program.

8. The system according to claim 7, wherein the predefined program defines steps executed by the surgical module to perform the surgical procedure.

9. The system according to claim 7, wherein the predefined program defines steps executed by a plurality of surgical modules to perform the surgical procedure.

10. A computer implemented method for delivering surgical instruments to a surgical field, the method comprising:
inserting a guide wire into a patient;
attaching a surgical module to the guide wire, wherein the surgical module includes a channel for movably coupling the surgical module to the guide wire;
manipulating magnetic fields produced at at least one of the guide wire and the surgical module; and
moving the surgical module relative to the guide wire responsive to the act of manipulating the magnetic fields.

11. The method according to claim 10, wherein the act of inserting the guide wire includes inserting the guide wire into a natural body opening of a patient.

12. The method according to claim 10, wherein the act of manipulating the magnetic fields is executed by at a control unit configured to manipulate the magnetic fields of at least one of the guide wire and the surgical module.

13. The method according to claim 12, wherein moving the surgical module relative to the guide wire includes moving the surgical module forward, backwards, and around relative to the guide wire.

14. The method according to claim 12, wherein the method includes manipulating the magnetic fields responsive to wireless control signals.

15. The method according to claim 12, wherein the act of moving includes an act of positioning the surgical module at a surgical field within a patient.

16. The method according to claim 12, wherein the method further comprises performing, by the surgical module, a surgical procedure.

17. The method according to claim 16, wherein the surgical procedure is executed according to a predefined program.

18. The method according to claim 17, wherein the predefined program defines steps executed by at least one surgical module to perform the surgical procedure.

19. The method according to claim 17, wherein the predefined program defines steps executed by a plurality of surgical modules to perform the surgical procedure.

20. A non-transitory computer readable medium having stored thereon sequences of instruction for delivering surgical instruments to a surgical field, including instructions that when executed cause at least one processor of a computer system to:
manipulate magnetic fields produced at at least one of a guide wire and a surgical module;
move the surgical module relative to the guide wire responsive to the act of manipulating the magnetic fields; and
control, the surgical module, during a surgical procedure.
